(19) United States
(12) Patent Application Publication
(10) Pub. No.: US 2006/0052414 A1
(43) Pub. Date: Mar. 9, 2006

(54) Compositions and methods for treating or preventing Hepadnaviridae infection

(75) Inventor: Dominique Dugourd, Vancouver (CA)

Correspondence Address: DARBY & DARBY P.C., P. O. BOX 5257, NEW YORK, NY, 10150-5257, US

(73) Assignee: Migenix, Inc., Vancouver (CA)

(21) Appl. No.: 11/202,867

(22) Filed: Aug. 12, 2005

Related U.S. Application Data
(60) Provisional application No. 60/601,217, filed on Aug. 13, 2004.

(51) Int. Cl. A61K 31/4745

(52) U.S. Cl. 514/306

(57) ABSTRACT

The present disclosure relates generally to the use of certain castanospermine esters to treat or prevent infections caused by Hepadnaviridae, particularly infections caused by hepatitis B virus (HBV), and to the use of such compounds to examine the biological mechanisms of HBV infection.

### CROSS-REFERENCE TO RELATED APPLICATION

This application claims the benefit of U.S. Provisional Patent Application No. 60/601,217, filed Aug. 13, 2004, which provisional application is incorporated herein by reference in its entirety.

### TECHNICAL FIELD

The present disclosure relates generally to the treatment of infectious disease, and more specifically, to the use of certain castanospermine esters to treat or prevent infections caused by or associated with Hepadnaviridae, particularly infections caused by or associated with hepatitis B virus (HBV).

### BACKGROUND

Hepadnaviridae infections, such as those caused by human hepatitis B virus (HBV), are a leading cause of liver disease and are linked epidemiologically to more serious complications (such as cirrhosis and hepatocellular carcinoma (HCC)). Various immunomodulatory vaccines and nucleoside analogs have been used to treat HBV infections, but HBV infection remains a major public health problem worldwide. In fact, more than one billion people have been infected and more than 350 million people are chronic carriers of HBV (Lee, N. Eng. J. Med. 333:1733, 1999).

Currently, five monotherapies have been approved for the treatment of chronic HBV infection—interferon-α-2b (Intron® A); peginterferon-α-2a (Pegasys®); (−)2′,3′-dideoxy, 3′-thiacytidine (3TC or lamivudine; Epivir-HBV®), adefovir dipivoxil (Hepsera®); and entecavir (Baraclude™). Interferon-α (IFN-α—lymphoblastoid, recombinant or pegylated) is an immunomodulator that is used in an attempt to achieve sustained suppression of HBV replication, as well as remission of HBV-related chronic liver disease (see, e.g., Karayiannis, J. Antimicrobial. Chemother. 51:761, 2003). However, IFN-α has numerous unwanted side-effects, including flu-like symptoms, malaise, depression, leucopenia, thrombocytopenia and thyroid dysfunction. Lamivudine, entecavir and adefovir dipivoxil are nucleoside analogs that inhibit viral replication, but extended use of these compounds can lead to toxicity and drug resistant HBV. Although no combination therapy has been approved for the treatment of HBV to date, a few studies have been conducted on combination therapies (e.g., IFN-α and a nucleoside analog, or two different nucleoside analogs together), but no definite conclusions on the efficacy of a combination therapy are possible because the results have been variable (Papatheodoridis and Hadziyannis, Ailment Pharmacol. Ther. 19:25, 2004).

Hence, a need exists for identifying and developing anti-Hepadnaviridae agents having improved activity and reduced toxicity, and in particular therapeutics for the treatment of HBV. The present invention meets such needs, and further provides other related advantages.

### SUMMARY

The present invention generally provides castanospermine derivatives, in particular, ester derivatives, and compositions of such compounds for use in treating or preventing, for example, Hepadnaviridae viral infections such as those caused by hepatitis B virus (HBV). In particular, the present disclosure provides castanospermine derivatives, either alone or in combination with other anti-Hepadnaviridae compounds, having unexpectedly high or synergistic inhibitory activity against Hepadnaviridae, such as HBV, respectively.

In one aspect, the instant disclosure provides methods of treating or preventing a Hepadnaviridae infection, comprising administering to a subject a glucosidase inhibitor and pharmaceutically acceptable salts thereof, wherein the glucosidase inhibitor or a pharmaceutically acceptable salt or derivative thereof has the following structural formula (I):

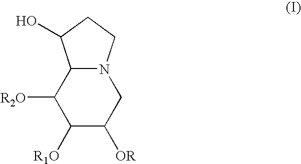

wherein R, R₁ and R₂ are independently hydrogen, C₁₋₁₄ alkanoyl, C₂₋₁₄ alkenoyl, cyclohexanecarbonyl, C₁₋₈ alkoxyacetyl,

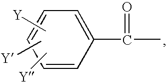

naphthalenecarbonyl optionally substituted by methyl or halogen; phenyl(C₂₋₆ alkanoyl) wherein the phenyl is optionally substituted by methyl or halogen; cinnamoyl; pyridinecarbonyl optionally substituted by methyl or halogen; dihydropyridine carbonyl optionally substituted by C₁₋₁₀ alkyl; thiophenecarbonyl optionally substituted by methyl or halogen; or furancarbonyl optionally substituted by methyl or halogen; Y is hydrogen, C₁₋₄ alkyl, C₁₋₄ alkoxy, halogen, trifluoromethyl, C₁₋₄ alkylsulfonyl, C₁₋₄ alkylmercapto, cyano or dimethylamino; Y′ is hydrogen, C₁₋₄ alkyl, C₁₋₄ alkoxy, halogen or it is combined with Y to give 3,4-methylenedioxy; Y″ is hydrogen, C₁₋₄ alkyl, C₁₋₄ alkoxy or halogen; wherein at least one, but not more than two, of R, R₁ and R₂ is hydrogen. In another embodiment, the glucosidase inhibitor structural formula (I) has the following stereochemistry:

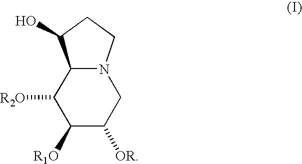

In certain embodiments, the glucosidase inhibitor is (a) [1S-(1α,6β,7α,8β,8aβ)]-octahydro-1,6,7,8-indolizinetetrol 6-benzoate; (b) [1S-(1α,6β,7α,8β,8aβ)]-octahydro-1,6,7,8-indolizinetetrol 7-benzoate; (c) [1S-(1α,6β,7α,8β,8aβ)]-octahydro-1,6,7,8-indolizinetetrol 6-(4-methylbenzoate); (d) [1S-(1α,6β,7α,8β,8aβ)]-octahydro-1,6,7,8-indolizinetetrol 7-(4-bromobenzoate); (e) [1S-(1α,6β,7α,8β,8aβ)]-octahydro-1,6,7,8-indolizinetetrol 6,8-dibutanoate; (f) [1S-(1α,6β,7α,8β,8aβ)]-octahydro-1,6,7,8-indolizinetetrol 6-butanoate; (g) [1S-(1α,6β,7α,8β,8aβ)]-octahydro-1,6,7,8-indolizinetetrol 6-(2-furancarboxylate); (h) [1S-(1α,6β,7α,8β,8aβ)]-octahydro-1,6,7,8-indolizinetetrol 7-(2,4-dichlorobenzoate); (j) [1S-(1α,6β,7α,8β,8aβ)]-octahydro-1,6,7,8-indolizinetetrol 6-(3-hexenoate); (k) [1S-(1α,6β,7α,8β,8aβ)]-octahydro-1,6,7,8-indolizinetetrol 6-octanoate; (l) [1S-(1α,6β,7α,8β,8aβ)]-octahydro-1,6,7,8-indolizinetetrol 6-pentanoate; (m) an O-pivaloyl ester; (n) a 2-ethyl-butyryl ester; (o) a 3,3-dimethylbutyryl ester; (p) a cyclopropanoyl ester; (q) a 4-methoxybenzoate ester; (r) a 2-aminobenzoate ester; or (s) a mixture of at least two of (a)-(q).

In another aspect, the instant disclosure provides a method for treating a Hepadnaviridae infection, comprising administering to a subject an agent that alters Hepadnaviridae replication, and a glucosidase inhibitor as described herein and pharmaceutically acceptable salts thereof, wherein the glucosidase inhibitor has a structure according to formula (I) as described herein. In certain embodiments, the subject is a non-human animal or a human.

In yet another aspect, the instant disclosure provides a method for treating or preventing a Hepadnaviridae infection, comprising administering to a subject a combination of an agent that alters immune function against Hepadnaviridae, and a glucosidase inhibitor and pharmaceutically acceptable salts thereof, wherein the glucosidase inhibitor has a structure according to formula (I) as described herein.

In a further aspect, the instant disclosure provides a method for treating or preventing Hepadnaviridae infection comprising a glucosidase inhibitor as described herein in combination with an agent selected from (a) a compound that inhibits infection of cells by Hepadnaviridae; (b) a compound that inhibits the release of viral RNA from the viral capsid or the function of Hepadnaviridae gene products; (c) a compound that alters Hepadnaviridae replication; (d) a compound that alters immune function against Hepadnaviridae; and (e) a compound that alters symptoms of a Hepadnaviridae infection.

In still another aspect, the instant disclosure provides a pharmaceutical composition comprising any of the compounds described herein, alone or in combination, and a pharmaceutically acceptable carrier, excipient or diluent for use in any of the methods described herein. In other embodiments, the composition further comprises an adjuvant, such as alum. In another embodiment, the composition further comprises other antimicrobial agents, such as one or more antibiotic, antifungal, anti-inflammatory, immunomodulatory, and other anti-viral compounds. Exemplary anti-viral compounds include adefovir dipivoxil, lamivudine, clevudine and ribavirin. Exemplary compounds that alter immune function include interferon, such as interferon-α, pegylated interferon-α, interferon-β, interferon-γ and cytokines. In certain embodiments, the compounds or compositions are administered orally, topically, or systemically. In still another embodiment, the composition is administered to a human to treat or prevent a Hepadnaviridae infection wherein the Hepadnaviridae is a member of the genus Avihepadnavirus or Orthohepadnavirus. In a related embodiment, the infection being treated is caused by or associated with hepatitis B virus (HBV), a Hepadnaviridae. In certain embodiments, the adjunctive therapeutic agent, such as an agent that alters viral replication or immune function, is administered before the glucosidase inhibitor, or the glucosidase inhibitor is administered before the adjunctive therapeutic agent, or the glucosidase inhibitor and the adjunctive therapeutic agent are admixed as a single composition and administered simultaneously.

### BRIEF DESCRIPTION OF THE DRAWINGS

FIGS. 1A-1C show graphs of the Fractional Effect (fraction of virus affected, i.e., the antiviral effect) plotted against the Combination Index (CI) to analyze synergism, additivity, or antagonism for combination therapies (two or more drugs). The data were analyzed with the CalcuSyn™ program (Biosoft, Inc.). A CI (shown as a straight line at 1.0) greater than 1.0 indicates antagonism, a CI of 1.0 indicates additivity, and a CI of less than 1.0 indicates synergism. A Monte Carlo analysis was included, which provides a measure of statistical significance, represented by the triple lines (the median value (middle line) with the upper and lower lines showing the SD, i.e., median values ±1.96 SDs).

FIGS. 2A-2C show isobolograms that include the predicted ED₅₀, ED₇₅, and ED₉₀ values (EM) for the combined drugs (celgosivir and adefovir dipivoxil) if they were to have an additive interaction. The actual ED₅₀, ED₇₅, and ED₉₀ values (μM) for the combination treatment of celgosivir and adefovir dipivoxil are displayed as single points. A single point value that falls to the right of the theoretical additive line indicates antagonism, and a value to the left of the additive line indicates synergy.

### DETAILED DESCRIPTION

As set forth above, the present invention provides compositions and methods for using and making glucosidase inhibitors and derivatives thereof, to treat or prevent infectious diseases, such as those caused by Hepadnaviridae. In particular, these glucosidase inhibitors and derivatives thereof are useful for treating or preventing viral infections, such as hepatitis B virus (HBV) infections. The invention, therefore, relates generally to the surprising discovery that certain glucosidase inhibitors and derivatives thereof have an unexpectedly high activity against Hepadnaviridae, such as HBV. Accordingly, the compounds of the invention are useful, for example, as research tools for in vitro and cell-based assays to study the biological mechanisms of HBV infection (e.g., replication and transmission), and are useful as potential therapeutics for the prevention or treatment of HBV infection and HBV-related disease. Discussed in more detail below are glucosidase inhibitors and derivatives thereof suitable for use within the present invention, as well as representative compositions and therapeutic uses.

By way of background, HBV is the prototype member of the viral family Hepadnaviridae. The family is comprised of two genera, namely Orthohepadnavirus and Avihepadnavirus, with the former being responsible for mammalian infections. HBV can be further classified into eight major genotypes (A-H) based on nucleotide diversity of ≧8%, with each genotype having a distinct global geographical distribution (Locarnini, Seminars in Liver Disease 24 (Supp. 1):3, 2004). The HBV genome is a 3.2 kb partially double-stranded, relaxed circular DNA (rcDNA) that is organized into four open-reading frames and is produced through reverse transcription of an RNA intermediate. The open reading frames encode a viral polymerase, envelope, core, and X proteins. Once inside the host nucleus, the partially double-stranded viral DNA is converted by the host cell into covalently closed circular DNA (cccDNA), from which four sets of mRNA are transcribed. The RNA encodes HBV core antigen (HBcAg or nucleocapsid protein), the soluble and secreted HBeAg, the Pol protein, the viral envelope proteins that includes HBsAg (the surface antigen that is diagnostic of a chronic HBV infection if it is detectable for 6 months), and HBV X protein. The focus has been on identifying inhibitors of specific cellular targets and processes involving nucleosides, such as inhibition of priming of reverse transcription, inhibition of the viral minus strand DNA elongation, and inhibition of the HBV polymerase. HBV contains three surface proteins (HBs proteins) of different sizes that are singly or doubly N-glycosylated and are essential for the formation of infectious virus.

Prior to setting forth the invention in more detail, it may be helpful to an understanding thereof to set forth definitions of certain terms to be used hereinafter.

In the present description, any concentration range, percentage range, or integer range is to be understood to include the value of any integer within the recited range and, when appropriate, fractions thereof (such as one tenth and one hundredth of an integer), unless otherwise indicated. As used herein, “about” or “comprising essentially of” mean ±15% of any indicated value, range or structure. The use of the alternative (e.g., “or”) should be understood to mean either one, both, or any combination thereof of the alternatives. In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present invention.

As used herein, the term “alkyl” refers to a saturated or unsaturated, branched, straight-chain or cyclic monovalent hydrocarbon group derived by the removal of one hydrogen atom from a single carbon atom of a parent alkane, alkene or alkyne. Representative alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl , prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; or the like.

The term “alkyl” is specifically intended to include straight- or branched-hydrocarbons having from 1 to 25 carbon atoms, or 5 to 20, or 10 to 18, or 1 to 5. The alkyls may have any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. When a specific level of saturation is intended, the expressions “alkanyl,” “alkenyl,” and “alkynyl” are used. The expression “lower alkyl” refers to alkyl groups comprising from 1 to 8 carbon atoms. The alkyl group may be substituted or unsubstituted.

“Alkanyl” refers to a saturated branched, straight-chain or cyclic alkyl group. Representative alkanyl groups include methanyl; ethanyl; propanyls such as propan-1-yl, propan-2-yl (isopropyl), cyclopropan-1-yl, etc.; butyanyls such as butan-1-yl, butan-2-yl (sec-butyl), 2-methyl-propan-1-yl (isobutyl), 2-methyl-propan-2-yl (t-butyl), cyclobutan-1-yl, etc.; or the like.

“Alkenyl” refers to an unsaturated branched, straight-chain, cyclic alkyl group, or combinations thereof having at least one carbon-carbon double bond derived by the removal of one hydrogen atom from a single carbon atom of a parent alkene. The group may be in either the cis or trans conformation about the double bond(s). Representative alkenyl groups include ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl , but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; or the like. The alkenyl group may be substituted or unsubstituted.

“Alkynyl” refers to an unsaturated branched, straight chain or cyclic alkyl group having at least one carbon-carbon triple bond derived by the removal of one hydrogen atom from a single carbon atom of a parent alkyne. Representative alkynyl groups include ethynyl; propynyls such as prop-1-yn-1-yl, prop-2-yn-1-yl, etc.; butynyls such as but-1-yn-1-yl, but-1-yn-3-yl, but-3-yn-1-yl, etc.; or the like.

“Alkyldiyl” refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Representative alkyldiyl groups include methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; or the like. When a specific level of saturation is intended, the nomenclature alkanyldiyl, alkenyldiyl or alkynyldiyl is used. In certain embodiments, the alkyldiyl group is (C₁-C₄) alkyldiyl. In further embodiments, saturated the alkyldiyl group is acyclic alkanyldiyl groups in which the radical centers are at the terminal carbons, e.g., methandiyl (methano); ethan-1,2-diyl (ethano); propan-1,3-diyl (propano); butan-1,4-diyl (butano); or the like (also referred to as alkylenos, defined infra).

“Alkyleno” refers to a straight-chain alkyldiyl group having two terminal monovalent radical centers derived by the removal of one hydrogen atom from each of the two terminal carbon atoms of straight-chain parent alkane, alkene or alkyne. Representative alkyleno groups include methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, but[1,3]diyno, etc.; or the like. When a specific level of saturation is intended, the nomenclature alkano, alkeno or alkyno is used. In certain embodiments, the alkyleno group is (C₁-C₆) or (C₁-C₄) alkyleno. In further embodiments, alkyleno group is a straight-chain saturated alkano group, e.g., methano, ethano, propano, butano, or the like.

“Heteroalkyl, Heteroalkanyl, Heteroalkenyl, Heteroalkanyl, Heteroalkyldiyl and Heteroalkyleno” refer to alkyl, alkanyl, alkenyl, alkynyl, alkyldiyl and alkyleno groups, respectively, in which one or more of the carbon atoms (and any associated hydrogen atoms) are each independently replaced with the same or different heteroatoms or heteroatomic groups. Representative heteroatoms or heteroatomic groups that can be included in these groups include —O—, —S—, —Se—, —O—O—, —S—S—, —O—S—, —O—S—O—, —O—NR′—, —NR′—, —NR′—NR′—, ═N—N═, —N═N—, —N═N—NR′—, —PH—, —P(O)₂—, —O—P(O)₂—, —SH₂—, —S(O)—, —SnH₂— and the like, and combinations thereof, including —NR′—S(O)₂—, where each R′ is independently selected from hydrogen, alkyl, alkanyl, alkenyl, alkynyl, aryl, arylalkyl, heteroaryl and heteroarylalkyl, as defined herein.

“Aryl” refers to a monovalent aromatic hydrocarbon group derived by the removal of one hydrogen atom from a single carbon atom of a parent aromatic ring system. Representative aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, or the like. In certain embodiments, the aryl group is (C₅-C₁₄) aryl, with (C₅-C₁₀) being even more preferred. In other embodiments, aryls are cyclopentadienyl, phenyl and naphthyl. The aryl group may be substituted or unsubstituted.

“Arylalkyl” refers to an acyclic alkyl group in which one of the hydrogen atoms bonded to a carbon atom, typically a terminal or sp³ carbon atom, is replaced with an aryl group. Representative arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl or the like. Where specific alkyl moieties are intended, the nomenclature arylalkanyl, arylakenyl or arylalkynyl is used. In certain embodiments, the arylalkyl group is (C₆-C₂₀) arylalkyl, e.g., the alkanyl, alkenyl or alkynyl moiety of the arylalkyl group is (C₁-C₆) and the aryl moiety is (C₅-C₁₄). In further embodiments, the arylalkyl group is (C₆-C₁₃), e.g., the alkanyl, alkenyl or alkynyl moiety of the arylalkyl group is (C₁-C₃) and the aryl moiety is (C₅-C₁₀).

“Heteroaryl” refers to a monovalent heteroaromatic group derived by the removal of one hydrogen atom from a single atom of a parent heteroaromatic ring system, which may be monocyclic or fused ring (i.e., rings that share an adjacent pair of atoms). Representative heteroaryl groups include groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, or the like. In certain embodiments, the heteroaryl group is a 5-14 membered heteroaryl or a 5-10 membered heteroaryl. In further embodiments, heteroaryl groups are those derived from thiophene, pyrrole, benzothiophene, benzofuran, indole, pyridine, quinoline, imidazole, oxazole or pyrazine. The heteroaryl group may be substituted or unsubstituted.

“Heteroalicyclic” refers to a monocyclic or fused ring group having in the ring(s) one or more atoms selected preferably from nitrogen, oxygen and sulfur. The rings may also have one or more double bonds. However, the rings do not have a completely conjugated π-electron system. The heteroalicyclic ring may be substituted or unsubstituted. When substituted, the substituted group(s) are independently selected from alkyl, aryl, haloalkyl, halo, hydroxy, alkoxy, mercapto, cyano, sulfonamidyl, aminosulfonyl, acyl, acyloxy, nitro, or substituted amino.

“Heteroarylalkyl” refers to an acyclic alkyl group in which one of the hydrogen atoms bonded to a carbon atom, typically a terminal or sp³ carbon atom, is replaced with a heteroaryl group. When one or more specific alkyl moiety is intended, the nomenclature heteroarylalkanyl, heteroarylakenyl or heterorylalkynyl is used. In certain embodiments, the heteroarylalkyl group is a 6-20 membered heteroarylalkyl, e.g., the alkanyl, alkenyl or alkynyl moiety of the heteroarylalkyl is 1-6 membered and the heteroaryl moiety is a 5-14-membered heteroaryl. In other embodiments, the heteroarylalkyl is a 6-13 membered heteroarylalkyl, e.g., the alkanyl, alkenyl or alkynyl moiety is 1-3 membered and the heteroaryl moiety is a 5-10 membered heteroaryl.

The various naphthalenecarbonyl, pyridinecarbonyl, thiophenecarbonyl and furancarbonyl groups referred herein include the various position isomers and these can be naphthalene-1-carbonyl, naphthalene-2-carbonyl, nicotinoyl, isonicotinoyl, N-methyl-dihydro-pyridine-3-carbonyl, thiophene-2-carbonyl, thiophene-3-carbonyl, furan-2-carbonyl and furan-3-carbonyl. The naphthalene, pyridine, thiophene and furan groups can be optionally further substituted as indicated herein.

“Halogen” or “halo” refers to fluoro (F), chloro (Cl), bromo (Br), iodo (I). As used herein, —X refers to independently any halogen.

“Acyl” group refers to the C(O)-R″ group, where R″ is selected from hydrogen, hydroxy, alkyl, haloalkyl, cycloalkyl, aryl optionally substituted with one or more alkyl, haloalkyl, alkoxy, halo and substituted amino groups, heteroaryl (bonded through a ring carbon) optionally substituted with one or more alkyl, haloalkyl, alkoxy, halo and substituted amino groups and heteroalicyclic (bonded through a ring carbon) optionally substituted with one or more alkyl, haloalkyl, alkoxy, halo and substituted amino groups. Acyl groups include aldehydes, ketones, acids, acid halides, esters and amides. Preferred acyl groups are carboxy groups, e.g., acids and esters. Esters include amino acid ester derivatives. The acyl group may be attached to a compound's backbone at either end of the acyl group, i.e., via the C or the R″. When the acyl group is attached via the R″, then C will bear another substituent, such as hydrogen, alkyl, heteroaryl or the like.

“Substituted” refers to a group in which one or more hydrogen atoms are 1 5 each independently replaced with the same or different substituent(s). Typical substituents include —X, —R¹³, —O—, ═O, —OR, —SR¹³, —S—, ═S, —NR¹³R¹³, ═N¹³, CX₃, —CF₃, —CN, —OCN, —SCN, —NO, NO₂, ═N₂, —N₃, —S(O)₂O—, —S(O)₂OH, —S(O)₂R¹³, —OS(O₂)O—, —OS(O)₂OH, —OS(O)₂R¹³, —P(O)(O⁻)₂, —P(O)(OH)(O⁻), —OP(O)₂(O⁻), —C(O)R¹³, —C(S)R¹³, —C(O)OR¹³, —C(O)O—, —C(S)OR¹³, and —C(NR¹³)NR¹³R¹³, wherein each X is independently a halogen; each R¹³ is independently hydrogen, halogen, alkyl, aryl, arylalkyl, arylaryl, arylheteroalkyl, heteroaryl, heteroarylalkyl NR¹⁴R¹⁴, —C(O)R¹⁴, and —S(O)₂R¹⁴; and each R¹⁴ is independently hydrogen, alkyl, alkanyl, alkynyl, aryl, arylalkyl, arylheteralkyl, arylaryl, heteroaryl or heteroarylalkyl.

“Prodrug” herein refers to a compound that is converted into the parent compound in vivo. Prodrugs often are useful because, in some situations, they may be easier to administer than the parent compound. For example, the prodrug may be more bioavailable by oral administration or for cellular uptake than a parent compound. The prodrug may also have improved solubility in pharmaceutical compositions over the parent compound. An example of a prodrug would be a compound of the embodiments of the present invention that is administered, for example, as an ester (the “prodrug”) to facilitate transmittal across a cell membrane when water solubility is detrimental to mobility, but then is metabolically hydrolyzed to an active entity once inside the cell where water solubility is beneficial. Such a compound is generally inactive (or less active) until converted to the active form.

“Pharmaceutically acceptable salt” refers to a salt of a compound of the invention that is pharmaceutically acceptable and that possesses the desired pharmacological (e.g., antiviral) activity. Such salts include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

Castanospermine and Derivatives Thereof

As set forth above, the present disclosure provides castanospermine derivatives, pharmaceutically acceptable salts thereof, and uses thereof. In particular, the castanospermine derivatives are esters. By way of background, a number of strategies have been used in the attempt to treat chronic HBV infection, wherein a treatment can include roughly achieving the following three goals: (1) elimination of infectivity and transmission of HBV to others, (2) arresting the progression of liver disease and improving clinical prognosis, or (3) preventing development of cirrhosis and HCC. To date, a therapeutic agent that adequately treats or prevents an HBV infection and any associated disease has remained elusive. The instant disclosure provides certain castanospermine ester derivatives that have unexpectedly high antiviral activity, and in particular high antiviral activity against HBV.

Castanospermine and certain imino sugars, such as deoxynojirimycin (DNJ), are ER α-glucosidase inhibitors, and both inhibit the early stages of glycoprotein processing. However, their effects differ substantially depending on the system to which they are applied and they appear to exhibit quite different specificities, castanospermine being relatively specific for α-glucosidase I.

Castanospermine is an alkaloid, originally isolated from the seeds of Castanospermum australe, having the following formula:

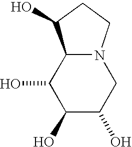

Systematically, this compound can be named in several ways as follows: [1S-(1α,6β,7α,8β,8aβ)]-octahydro-1,6,7,8-indoli-zinetetrol or [1S,(1S,6S,7R,8R,8aR)-1,6,7,8-tetrahydroxy-indolizidine or 1,2,4,8-tetradeoxy-1,4,8-nitrilo-L-glycero-D-galacto-octitol. The term “castanospermine” or the first listed systematic name will be used herein.

The esters of the present disclosure are prepared by the reaction of castanospermine with an appropriate acid chloride or anhydride in an inert solvent. The halide can be a chloride or bromide and the anhydride includes mixed anhydrides. The relative amount of the acid halide or anhydride used, the relative amount of solvent, the temperature and the reaction time are all controlled so as to minimize the number of hydroxy groups that will be acylated. Thus, only a limited excess of the acid derivative is used, which means up to about a three-fold excess of the acylating agent.

Use of a solvent in relatively large amounts, serves to dilute the reactants and suppress the amount of higher acylated products that form. The solvent used is preferably one that will dissolve the reactants used without reacting with them.

In certain embodiments, it may be advantageous to carry out the reaction in the presence of a tertiary amine which will react with and remove any acid formed during the course of the reaction. The tertiary amine can be added to the mixture or it can itself be used in excess and serve as the solvent. For example, pyridine can be used. As indicated herein, the time and the temperature are likewise controlled to limit the amount of acylation that takes place. Preferably, the reaction is carried out with cooling in an ice-bath for a period of about 16 hours to give the monoesters with the reaction time extended to a longer period, such as 7 days, if diesters are desired. The reaction can actually be carried out at higher temperatures and heating can be used as long as the various factors involved are properly controlled.

When the reaction is carried out as described herein, the final reaction mixture may still contain a considerable amount of unreacted castanospermine. This unreacted material can be recovered from the reaction mixture and recycled in subsequent reactions and, therefore, increase the overall amount of castanospermine converted to an ester. This recycling is particularly important when the reaction is carried out under conditions that would favor the isolation of monoesters. The procedures as described herein will generally yield 6- or 7-monoesters or 6,7- or 6,8-diesters. Other isomers can be obtained by appropriate use of blocking groups. For example, castanospermine can be reacted with 2-(dibromomethyl)benzoyl chloride to give the 6,7-diester. This diester is then reacted with an appropriate acid halide or anhydride to give the corresponding 8-ester. The two protecting groups are then readily removed by conversion of the two dibromomethyl groups to formyl (using silver perchlorate and 2,4,6-collidine in aqueous acetone) followed by hydrolysis of the formylbenzoic acid ester obtained using morpholine and hydroxide ion. The indicated procedure can be used in a similar way to give diester isomers.

With 1,8-O-isopropylidene castanospermine or 1,8-cyclohexylidene castanospermine, the reaction with an acid chloride in a standard esterification procedure favors the formation of the 6-ester almost exclusively. The isopropylidene or cyclohexylidene group is then removed by treatment with an acid such as 4-toluenesulfonic acid. The starting ketal compounds are themselves obtained form castanospermine 6,7-dibenzoate. This dibenzoate is reacted with 2-methoxypropene or 1-methoxycyclohexene and acid to introduce the 1,8-O-isopropylidene or 1,8-O-cyclohexylidene group and the two benzoate ester groups are removed by hydrolysis with base such as sodium hydroxide or by transesterification with sodium or potassium alkoxide as the catalyst.

In certain embodiments, the present disclosure provides methods of treating or preventing a Hepadnaviridae infection, comprising administering to a subject a glucosidase inhibitor and pharmaceutically acceptable salts thereof, wherein the glucosidase inhibitor has the following structural formula (I):

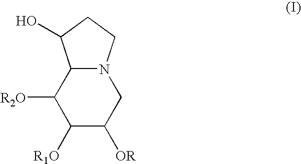

wherein R, R₁ and R₂ are independently hydrogen, C₁₋₁₄ alkanoyl, C₂₋₁₄ alkenoyl, cyclohexanecarbonyl, C₁₋₈ alkoxyacetyl,

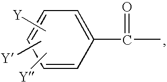

naphthalenecarbonyl optionally substituted by methyl or halogen; phenyl(C₂₋₆ alkanoyl) wherein the phenyl is optionally substituted by methyl or halogen; cinnamoyl; pyridinecarbonyl optionally substituted by methyl or halogen; dihydropyridine carbonyl optionally substituted by C₁₋₁₀ alkyl; thiophenecarbonyl optionally substituted by methyl or halogen; or furancarbonyl optionally substituted by methyl or halogen; Y is hydrogen, C₁₋₄ alkyl, C₁₋₄ alkoxy, halogen, trifluoromethyl, C₁₋₄ alkylsulfonyl, C₁₋₄ alkylmercapto, cyano or dimethylamino; Y′ is hydrogen, C₁₋₄ alkyl, C₁₋₄ alkoxy, halogen or it is combined with Y to give 3,4-methylenedioxy; Y″ is hydrogen, C₁₋₄ alkyl, C₁₋₄ alkoxy or halogen; with R, R₁ and R₂ being selected in such a way that at least one of them, but not more than two of them, is hydrogen; or a pharmaceutically acceptable salt or derivative thereof. In another embodiment, the glucosidase inhibitor structural formula (I) has the following stereochemistry:

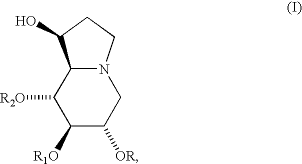

In certain embodiments, the castanospermine esters have the structures shown in Table 1.

TABLE 1

| Compound | R | Compound | R |
|---|---|---|---|
| CAST | H | MDL 29270 | H |
| MDL 28574 | CH₃(CH₂)₂—CO— | MDL 44370 | 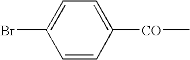 |
| MDL 43305 | 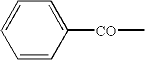 | MDL 29797 | CH₃(CH₂)₆—CO— |
| MDL 28653 | 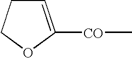 | MDL 29710 | CH₃(CH₂)₃—CO— |
| MDL 29435 | 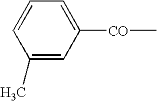 | MDL 29513 | CH₃ CH₂ (CH₂)₂ CH₂—CO— |
| MDL 29204 | 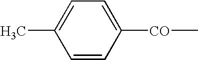 | | |

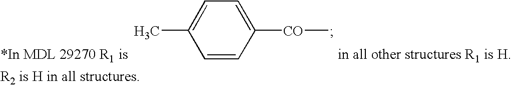

In further embodiments, provided are castanospermine esters of structure (I) wherein R₁ is a C₁₋₈ alkanoyl, C₂₋₁₀ alkenoyl, C₁₋₈ alkoxy-acetyl, or a benzoyl optionally substituted with an alkyl or halogen group. R₁ may be a C₁₋₈ alkanoyl, C₂₋₈ alkenoyl, C₁₋₈ alkoxyacetyl, or a benzoyl optionally substituted with a methyl, bromo, chloro, or fluoro group.

In certain embodiments, the glucosidase inhibitor is (a) [1S-(1α,6β,7α,8β,8aβ)]-octahydro-1,6,7,8-indolizinetetrol 6-benzoate; (b) [1S-(1α,6β,7α,8β,8aβ)]-octahydro-1,6,7,8-indolizinetetrol 7-benzoate; (c) [1S-(1α,6β,7α,8β,8aβ)]-octahydro-1,6,7,8-indolizinetetrol 6-(4-methylbenzoate); (d) [1S-(1α,6β,7α,8β,8aβ)]-octahydro-1,6,7,8-indolizinetetrol 7-(4-bromobenzoate); (e) [1S-(1α,6β,7α,8β,8aβ)]-octahydro-1,6,7,8-indolizinetetrol 6,8-dibutanoate; (f) [1S-(1α,6β,7α,8β,8aβ)]-octahydro-1,6,7,8-indolizinetetrol 6-butanoate; (g) [1S-(1α,6β,7α,8β,8aβ)]-octahydro-1,6,7,8-indolizinetetrol 6-(2-furancarboxylate); (h) [1S-(1α,6β,7α,8β,8aβ)]-octahydro-1,6,7,8-indolizinetetrol 7-(2,4-dichlorobenzoate); (j) [1S-(1α,6β,7α,8β,8aβ)]-octahydro-1,6,7,8-indolizinetetrol 6-(3-hexenoate); (k) [1S-(1α,6β,7α,8β,8aβ)]-octahydro-1,6,7,8-indolizinetetrol 6-octanoate; (l) [1S-(1α,6β,7α,8β,8aβ)]-octahydro-1,6,7,8-indolizinetetrol 6-pentanoate; (m) an O-pivaloyl ester; (n) a 2-ethyl-butyryl ester; (o) a 3,3-dimethylbutyryl ester; (p) a cyclopropanoyl ester; (q) a 4-methoxybenzoate ester; (r) a 2-aminobenzoate ester; or (s) a mixture of at least two of (a)-(q). In a specific embodiment, the glucosidase inhibitor is [1S-(1α,6β,7α,8β,8aβ)]-octahydro-1,6,7,8-indolizinetetrol 6-butanoate (also referred to as celgosivir or BuCast).

“Structurally pure” refers to a compound composition in which a substantial percentage, e.g., on the order of 95% to 100%, of the individual molecules each contain the same number and types of atoms attached to each other in the same order and with the same bonds. In certain embodiments the structural purity ranges from about 95%, about 96%, about 97%, about 98%, about 99% or more. As used herein, “structurally pure” is not intended to distinguish different geometric isomers or different optical isomers from one another. For example, as used herein a mixture of cis- and trans-but-2,3-ene is considered structurally pure, as is a racemic mixture. When compositions are intended to include a substantial percentage of a single geometric isomer or optical isomer, the nomenclature “geometrically pure” and “optically or enantiomerically pure,” respectively, are used.

The phrase “structurally pure” is also not intended to discriminate between different tautomeric forms or ionization states of a molecule, or other forms of a molecule that result from equilibrium phenomena or other reversible interconversions. Thus, a composition of, for example, an organic acid is structurally pure even though some of the carboxyl groups may be in a protonated state (COOH) and others may be in a deprotonated state (COO⁻). Likewise, a composition comprising a mixture of keto and enol tautomers, unless specifically noted otherwise, is considered structurally pure.

Therapeutic Formulations and Methods of Use

As described herein, compounds of the instant disclosure are useful for treating or preventing Hepadnaviridae infection, particularly HBV. In certain embodiments, the disclosure provides compounds capable of treating or preventing Hepadnaviridae infection, such as HBV, at clinically relevant concentrations or by statistically measurable criteria.

Exemplary cell-based assays for the evaluation of antiviral activity against HBV are known in the art (see, e.g., Korba et al., Antiviral Res. 15:217, 1991; and Korba et al., Antiviral Res. 19:55, 1992). Moreover, the compounds of the instant disclosure can be useful research tools for in vitro and cell-based assays to study the biological mechanisms of viral infection, growth, and replication, such as for HBV. By way of background and not wishing to be bound by theory, HBV morphogenesis is complex wherein preassembled viral core particles are believed to attach to cytosolic sides of viral envelope (surface) proteins, which have inserted in the endoplasmic reticulum (ER) membrane. After acquiring envelopes, virions bud to the lumen of the ER and then are transported through the Golgi apparatus to the extracellular fluids. Removal of N-linked glucose residues (trimming is done by cellular enzymes, such as α-glucosidases) from immature viral glycoproteins may play a role in the migration and maturation of viral glycoproteins from the ER to the Golgi.

In one embodiment, the instant disclosure provides a method of identifying anti-viral compounds, comprising contacting a host cell infected with a virus (e.g., Hepadnaviridae, such as HBV) with a candidate castanospermine ester or derivative thereof of the instant disclosure for a time sufficient to alter viral morphogenesis or production, and identifying a candidate derivative that alters viral morphogenesis or production. In another embodiment, there is provided a method of identifying cells suspected of having a viral infection, comprising contacting a host cell suspected of being infected with a virus with a candidate castanospermine ester or derivative thereof of the invention for a time sufficient to alter viral morphogenesis or production, and thereby identifying cells infected with a virus. Preferably, the viral infection is caused by or associated with Hepadnaviridae, such as HBV.

In addition, in vivo models, such as woodchucks and Peking duck, for evaluating compounds for antiviral activity against HBV are known in the art (see, e.g., Tennant et al., ILAR Journal 42:89, 2001; Zuckerman, J. Virology Methods, 17:119, 1987; Aguesse-Germon et. al., Antimicrobial Agents and Chemotherapy 42:369, 1998). Furthermore, as a person having ordinary skill in the art would understand, these in vitro and in vivo assays can be used to determine the therapeutic value of a candidate compound and used to determine which dosage parameters would be most useful in treating a subject for a viral infection. In certain embodiments, the subject to be treated is a non-human animal or is a human.

The instant disclosure also relates to pharmaceutical compositions that contain one or more compounds (e.g., castanospermine esters) used for treating or preventing a viral infection (e.g., Hepadnaviridae, such as HBV). The invention further relates to methods for treating or preventing viral infections by administering to a subject a glucosidase inhibitor, or a cocktail mixture of two or more of such compounds, at a dose sufficient to treat or prevent a viral infection, as described herein. The castanospermine ester derivatives, or cocktails of such compounds, are preferably part of a pharmaceutical composition when used in the methods as described in the present disclosure.

In certain embodiments, the castanospermine esters and derivatives thereof described herein are used for treating or preventing a viral infection in a subject, such as a subject that is a non-human animal or a human. In other embodiments, the viral infection is due to Hepadnaviridae, HBV or other single-stranded DNA viruses. Certain compounds of the instant disclosure, including the compound derivatives of structure (I), show good overall biopharmaceutical properties and are orally available. In one embodiment, provided is a pharmaceutical composition comprising a glucosidase inhibitor as described herein (or a pharmaceutically active derivative thereof) and a pharmaceutically acceptable carrier, excipient or diluent for use in the methods of treatment described herein. In further embodiments, the pharmaceutical composition comprises an anti-viral compound (i.e., glucosidase inhibitor) that is a derivative of structure (I). The term “pharmaceutically active derivative” refers to any compound that, upon administration to a subject in need thereof, is capable of providing directly or indirectly (e.g., a pro-drug) the active compounds of the instant invention.

As set forth herein, the active compound may be included in a pharmaceutically acceptable carrier or diluent for administration to a subject in need thereof in an amount effective to treat or prevent a Hepadnaviridae infection, such as an HBV infection. Representative doses of the active compound for any of the indications mentioned herein can range from about 0.01 mg/kg to about 300 mg/kg per day, from about 0.1 mg/kg to about 100 mg/kg per day, or from about 0.5 mg/kg to about 25 mg/kg body weight of the recipient per day. Exemplary topical dosages will range from about 0.01-3% wt/wt in a suitable carrier. The effective dosage range of a pharmaceutically acceptable derivative can be calculated based on the molecular mass of the parent compound to be delivered. If the derivative exhibits activity in itself, the effective dosage can be estimated as above using the mass of the derivative, or by other means known to a person having ordinary skill in the art.

The active ingredient should be administered to achieve peak plasma concentrations of the active compound of about 0.001 μM to about 30 μM, or about 0.01 μM to about 10 μM. This may be achieved, for example, by intravenous injection of a composition or formulation of a castanospermine ester or derivative thereof of the instant disclosure, optionally in saline or other aqueous medium. In another embodiment, a castanospermine ester or derivative thereof of the invention or composition thereof is administered as a bolus.

The concentration of active compound in a pharmaceutical composition of the instant invention will depend on absorption, distribution, inactivation, and excretion rates of the drug, as well as other factors known to those of skill in the art. It is to be understood that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions or methods. The active ingredient may be administered all at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterores; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents. See, generally, “Remington's Pharmaceutical Sciences,” Mack Publishing Co., Easton, Pa. (A. R. Gennaro, ed., 18^{th} Edition, 1990).

The active compound or pharmaceutically acceptable salt or derivative thereof can be administered as a component of an elixir, suspension, syrup, wafer, chewing gum or the like. A syrup may contain, in addition to the active compounds, sucrose as a sweetening agent and certain preservatives, dyes and colorings and flavors.

The pharmaceutical composition of the instant invention can include at least one of a pharmaceutically acceptable vehicle, carrier, diluent, or excipient, in addition to one or more castanospermine ester or derivative thereof and, optionally, other components or active ingredients such as other anti-HBV drugs, including agents that alter viral replication (e.g., nucleoside analogs) or an immune response (e.g., interferons). Exemplary compositions of the instant disclosure include castanospermine esters or derivatives thereof, or a cocktail of two or more castanospermine esters or derivatives thereof, or a cocktail of one or more castanospermine esters or derivatives thereof with one or more antibiotic, antifungal, anti-inflammatory, immunomodulatory or other anti-viral compound as described herein (including interferons, cytokines, nucleoside analogs or the like).

The pharmaceutical compositions of the instant disclosure for use in treating Hepadnaviridae infections may also comprise glucosidase inhibitors having the structure of formula (I) in association (e.g., in admixture or co-packaged) with an adjunctive therapeutic. For example, provided is a composition comprising a castanospermine ester or castanospermine ester composition as described herein (including, e.g., [1S-(1α,6β,7α,8β,8aβ)]-octahydro-1,6,7,8-indolizinetetrol 6-butanoate), in combination with a compound that alters the binding to or infection of cells by Hepadnaviridae, including antibodies (e.g., monoclonal antibodies against, for example, HBcAg, HBeAg, HBsAg, and fragments or derivatives thereof; see, e.g., Valenzuela et al., Nature 298:347, 1982; Bitter et al., J. Med. Virol. 25:123, 1988; EP 0226846; EP 0299208; EP 0278940; U.S. Pat. Nos. 5,196,194; 5,369,637; 5,770,584; 6,100,065; 6,146,629; 6,410,009; 6,419,931; 6,488,934; 6,589,530; 6,589,534; 6,627,202; 6,680,053; 6,787,142; 6,924,368) and glucosaminoglycans (such as heparan and suramin).

The pharmaceutical compositions of the instant disclosure for use in treating Hepadnaviridae infections may also comprise glucosidase inhibitors having the structure of formula (I) in association (e.g., in admixture or co-packaged) with an adjunctive therapeutic. For example, provided is a composition comprising a castanospermine ester or castanospermine ester composition as described herein (including, e.g., [1S-(1α,6β,7α,8β,8aβ)]-octahydro-1,6,7,8-indolizinetetrol 6-butanoate), in combination with a compound that alters the release of viral RNA from the viral capsid or the function of Hepadnaviridae gene products, including inhibitors of the viral polymerase/replicase.

The pharmaceutical compositions of the instant disclosure for use in treating Hepadnaviridae infections may also comprise glucosidase inhibitors having the structure of formula (I) in association (e.g., in admixture or co-packaged) with an adjunctive therapeutic. For example, provided is a composition comprising a castanospermine ester or castanospermine ester composition as described herein (including, e.g., [1S-(1α,6β,7α,8β,8aβ)]-octahydro-1,6,7,8-indolizinetetrol 6-butanoate), in combination with a compound that alters Hepadnaviridae replication, or that alters cellular functions involved in or that influence Hepadnaviridae replication, including, e.g., 3′-thiacytidine (3TC or lamivudine), adefovir dipivoxil (Hepsera®), zidovudine, amdoxovir (DAPD), stavudine, didanosine, a carboxylic analog of 2′-deoxyguanosine such as entecavir (Baraclude(&), 1-[2-fluoro-5-methyl-β, L-arabinosyl] uracil (clevudine), famciclovir, penciclovir, heteroaryldihydropyrimidines (HAPs), β-L-nucleosides, 1,3-oxaselenolane nucleosides (see, e.g., U.S. Pat. No. 6,590,107), 2′,3′-dideoxy-2′,3′-didehydro-β-L-fluorocytidine, inhibitors of inosine monophosphate dehydrogenase (IMPDH) (such as ribavirin, mycophenolic acid, VX-497 (merimepodib)), tenofovir disoproxil fumarate, emtricitabine (FTC), telbivudine, elvucitabine, valtorcitabine, racivir, pradefovir and abacavir), see also, e.g., the compounds in U.S. Pat. No. 6,525,033; non-nucleoside RT inhibitors (e.g., efavirenz, nevirapine); protease inhibitors (e.g., saquinavir, indinavir and ritonavir); and other inhibitors of glycoprotein processing, such as DNJ and derivatives thereof.

The pharmaceutical compositions of the instant disclosure for use in treating Hepadnaviridae infections may also comprise glucosidase inhibitors having the structure of formula (I) in association (e.g., in admixture or co-packaged) with an adjunctive therapeutic. For example, provided is a composition comprising a castanospermine ester or castanospermine ester composition as described herein (including, e.g., [1S-(1α,6β,7α,8β,8aβ)]-octahydro-1,6,7,8-indolizinetetrol 6-butanoate), in combination with a compound that alters immune function (increase or decrease in a clinically significant or biologically significant manner) against Hepadnaviridae, for example, to stimulate a T cell response or to enhance a specific immune response (e.g., thymosin-α; cytokines; interferon, such as interferon-α, interferon-α2a (Roferon®-A), interferon-α2b (IntronA®), interferon-αcon-1 (Infergen®), interferon-β, interferon-β1a, interferon-β1b, interferon-γ, pegylated interferon (Pegasys®, Peg-Intron®; see also, e.g., U.S. Pat. Nos. 6,607,727; 6,689,363; 6,919,203).

The pharmaceutical compositions of the instant disclosure for use in treating Hepadnaviridae infections may also comprise glucosidase inhibitors having the structure of formula (I) in association (e.g., in admixture or co-packaged) with an adjunctive therapeutic. For example, provided is a composition comprising a castanospermine ester or castanospermine ester composition as described herein (including, e.g., [1S-(1α,6β,7α,8β,8aβ)]-octahydro-1,6,7,8-indolizinetetrol 6-butanoate), in combination with a compound that acts to modulate (preferably decrease or reduce the severity or intensity of, reduce the number of, or abrogate) symptoms and effects of Hepadnaviridae infection (e.g., antioxidants, such as the flavinoids).

In certain embodiments of any pharmaceutical composition or combination therapy described herein, the Hepadnaviridae infection to be treated is HBV. In further embodiments, the glucosidase inhibitor is [1S-(1α,6β,7α,8β,8aβ)]-octahydro-1,6,7,8-indolizinetetrol 6-butanoate. In further embodiments, the adjunctive therapeutic combined with a castanospermine ester is interferon, such as interferon-α or pegylated interferon-α. In certain embodiments, the adjunctive therapeutic combined with a castanospermine ester is a nucleoside analog, such as lamivudine, adefovir dipivoxil or clevudine. In further embodiments, the glucosidase inhibitor is [1S-(1α,6β,7α,8β,8aβ)]-octahydro-1,6,7,8-indolizinetetrol 6-butanoate and the adjunctive therapeutic is interferon, such as interferon-α or pegylated interferon-α. In further embodiments, the glucosidase inhibitor is [1S-(1α,6β,7α,8β,8aβ)]-octahydro-1,6,7,8-indolizinetetrol 6-butanoate and the adjunctive therapeutic is a nucleoside analog, such as lamivudine, adefovir dipivoxil or clevudine.

The adjunctive therapeutics discussed above can be administered together with the castanospermine esters of the disclosure, or the castanospermine esters and adjunctive therapeutic(s) can be sequentially administered, or any combination thereof. Methods for determining the effects of castanospermine esters or each adjunctive therapeutic (such as, for example, altering an immune response against Hepadnaviridae, modulating symptoms or effects of Hepadnaviridae infection, or altering Hepadnaviridae replication by, for example, adversely affecting, preventing, decreasing, or inhibiting viral replication), may be determined by methods routinely practiced by a skilled artisan (see, e.g., Korba et al., 1991, supra; Korba et al., 1992, supra; Locarnini, supra).

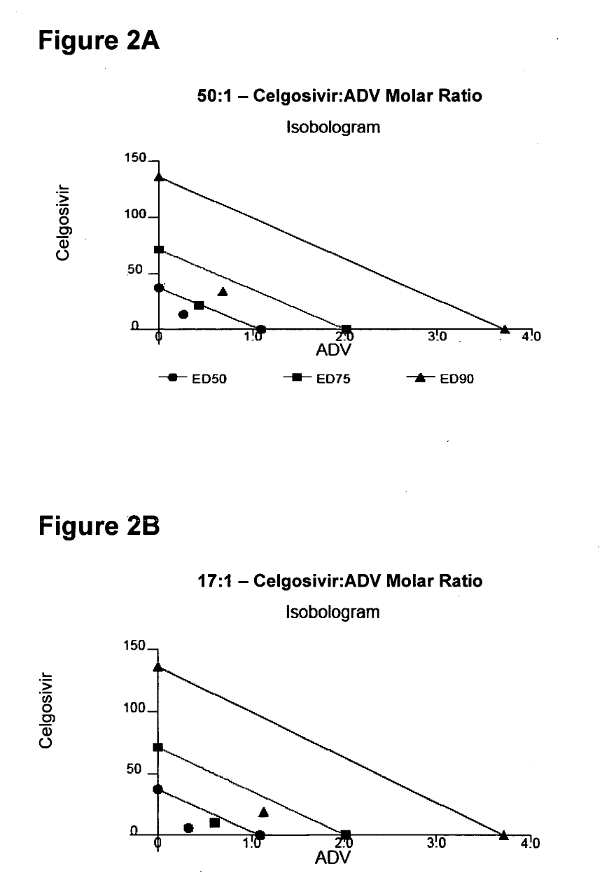

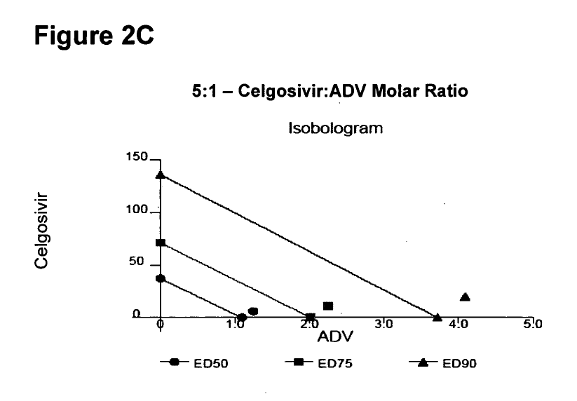

In one embodiment, a composition comprising a glucosidase inhibitor, an agent that alters immune function and an agent that alters viral replication will synergistically act to treat infection by Hepadnaviridae, such as HBV, in a subject. Two or more compounds that act synergistically interact such that the combined effect of the compounds is greater than the sum of the individual effects of each compound when administered alone (see, e.g., Berenbaum, Pharmacol. Rev. 41:93, 1989). For example, an interaction between a castanospermine ester or derivative thereof and another agent or compound may be analyzed by a variety of mechanistic and empirical models (see, e.g., Ouzounov et al., Antivir. Res. 55:425, 2002). A commonly used approach for analyzing interaction between a combination of agents employs the construction of isoboles (iso-effect curves, also referred to as isobolograms; see FIG. 2), in which the combination of agents (dₐ,d_{b}) is represented by a point on a graph, the axes of which are the dose-axes of the individual agents (see, e.g., Ouzounov et al., supra; see also Tallarida, J. Pharmacol. Exp. Therap. 298:865, 2001).

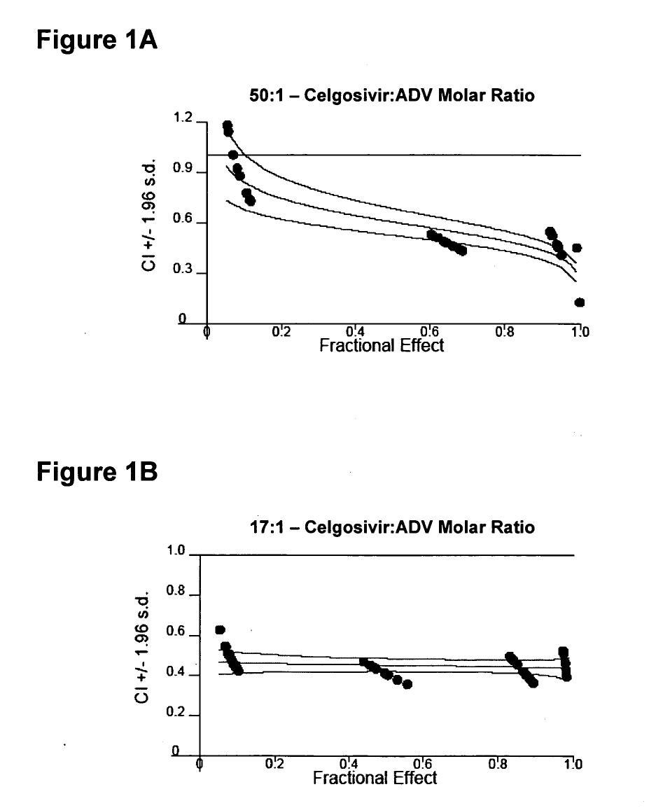

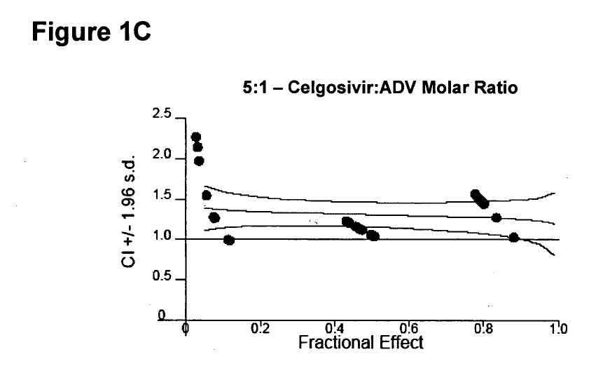

Another method for analyzing drug-drug interactions (antagonism, additivity, synergism) known in the art includes determination of combination indices (CI) according to the median effect principle to provide estimates of IC₅₀ values of compounds administered alone and in combination (see, e.g., Chou. In Synergism and Antagonism Chemotherapy. Eds. Chou and Rideout. Academic Press, San Diego Calif., pages 61-102, 1991; Okuse et al., Antiviral Res. 65:23, 2005; CalcuSyn™ software). A CI value of less than one represents synergistic activity, equal to one represents additive activity, and a value greater than one represents antagonism (see FIG. 1).

Pharmaceutically acceptable carriers suitable for use with a composition may include, for example, a thickening agent, a buffering agent, a solvent, a humectant, a preservative, a chelating agent, an adjuvant, and the like, and combinations thereof. Pharmaceutically acceptable carriers for therapeutic use are well known in the pharmaceutical art, and as described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992).

Solutions or suspensions used for parenteral, intradermal, subcutaneous, or topical application can include the following components: a sterile diluent such as water for injection, saline solution, fixed oils, polyethylene glycols, glycerin, propylene glycol or other synthetic solvents; anti-bacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. The parental preparation can be enclosed in ampoules, disposable syringes or multiple dose vials made of glass or plastic. If administered intravenously, preferred carriers are physiological saline or phosphate buffered saline (PBS) or an adjuvant. Exemplary adjuvants are alum (aluminum hydroxide, REHYDRAGEL®), aluminum phosphate, virosomes, liposomes with and without Lipid A, Detox (Ribi/Corixa), MF59, or other oil and water emulsions type adjuvants, such as nanoemulsions (see, e.g., U.S. Pat. No. 5,716,637) and submicron emulsions (see, e.g., U.S. Pat. No. 5,961,970), and Freund's complete and incomplete. In one preferred embodiment, a pharmaceutical composition of the invention is sterile.

In certain embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. For example, as is known in the art, some of these materials can be obtained commercially from Alza Corporation (CA) and Gilford Pharmaceuticals (Baltimore, Md.).

Liposomal suspensions may also be pharmaceutically acceptable carriers. These may be prepared according to methods known to those skilled in the art, for example, as described in U.S. Pat. No. 4,522,811. For example, liposome formulations may be prepared by dissolving appropriate lipid(s) (such as stearoyl phosphatidyl ethanolamine, stearoyl phosphatidylcholine, arachadoyl phosphatidylcholine, and cholesterol) in an inorganic solvent that is then evaporated, leaving behind a thin film of dried lipid on the surface of the container. An aqueous solution of the active compound or its monophosphate, diphosphate, or triphosphate derivative is then introduced into the container. The container is then swirled by hand to free lipid material from the sides of the container and to disperse lipid aggregates, thereby forming the liposomal suspension.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. The invention having been described, the following examples are intended to illustrate, and not limit, the invention.

### EXAMPLES

### Example 1

### Anti-HBV Activity of Castanospermine Esters with or without an Adjunctive Agent in an in vitro Culture Model

Confluent cultures of chronic HBV producing cell line HepG 2.2.15 in 96-well tissue culture plates in RPMI 1640 medium with 2% fetal bovine serum were used for the antiviral analysis of castanospermine esters (see Korba and Gerin, Antivir. Res. 19:55-70, 1992). Six cultures wells/each test concentration, in duplicate, were treated with 9 consecutive daily doses of celgosivir during which the medium was changed daily with the addition of fresh celgosivir. Daily aliquots of celgosivir or control compounds were frozen, and then thawed in culture medium at room temperature for immediate use. A positive antiviral control (lamivudine (3TC) or adefovir dipivoxil (ADV)) was included in each assay.

Extracellular HBV nucleic acid levels were measured 24 hours after the last treatment by Southern blot hybridization (quantified via densitometry using equipment and methods known in the art) as a measure of the number of HBV virions produced. Data are presented in Table 2. The level of extracellular virion DNA in untreated cells was 121 pg/mL culture medium. The sensitivity of the assay was 0.1 pg/mL.

TABLE 2
HBV Virion DNA Levels (pg/mL culture medium)

| Compound/Dose | 300 μM | 150 μM | 50 μM | 15 μM |
|---|---|---|---|---|
| Celgosivir | 8 ± 1 | 17 ± 2 | 56 ± 4 | 113 ± 9 |

| | 10 μM | 3.0 μM | 1.0 μM | 0.3 μM |
|---|---|---|---|---|
| ADV | 3 ± 1 | 11 ± 2 | 55 ± 4 | 121 ± 13 |

| | 150 μM | 50 μM | 15 μM | 5.0 μM | 1.5 μM | 0.5 μM |
|---|---|---|---|---|---|---|
| Celgosivir | 16 ± 2 | 53 ± 6 | 111 ± 8 | nd† | nd | nd |
| Celgosivir (50:1)* | 1 ± 1 | 8 ± 1 | 43 ± 5 | 126 ± 14 | 115 ± 15 | 123 ± 14 |
| Celgosivir (17:1)* | — | 3 ± 1 | 17 ± 2 | 65 ± 7 | 120 ± 13 | 117 ± 10 |
| Celgosivir (5:1)* | — | — | 26 ± 5 | 63 ± 5 | 116 ± 12 | 111 ± 12 |

*Combination treatment of celgosivir + ADV (molar ratio) − the μM dose value refers to the amount of celgosivir (e.g., Celgosivir (50:1) = 150 μM celgosivir: 3 μM ADV). If there is a “—”, then there was an undetectable level of HBV DNA.
†nd = not determined.

Based on the hybridization analysis, about 1.0 pg extracellular HBV DNA/ml of culture medium is roughly equivalent to about 3×10⁵ viral particles/ml. Values ±standard deviations (SD) were calculated by linear regression analysis using data combined from all treated cultures. SDs were calculated using the standard error of regression generated from the linear regression analyses. Using the results of experiments such as that presented in Table 2, the antiviral activity of celgosivir could be determined (see Table 3).

TABLE 3
Anti-HBV Activity of Celgosivir

| Compound | EC₅₀ (μM) | EC₉₀ (μM) |
|---|---|---|
| Celgosivir | 28 ± 1.4 | 158 ± 22 |
| ADV | 0.950 ± 0.105 | 2.8 ± 0.1 |
| 3TC | 0.058 ± 0.004 | 0.177 ± 0.015 |

EC₅₀ and EC₉₀ are a measure of antiviral activity, which indicate the drug concentration at which a 2-fold or a 10-fold reduction, respectively, in HBV DNA was observed as compared to the average level in untreated cultures. As shown in Tables 2 and 3, celgosivir caused a reduction in HBV virion production by the 2.2.15 cells at a clinically significant concentration. Moreover, the combination of celgosivir with ADV showed superior activity to either compound alone (and, in fact, this was surprisingly synergistic as discussed in Example 3).

### Example 2

### Cytotoxicity of Castanospermine Esters with or without an Adjunctive Agent in an in vitro Cell Culture Model

Cytotoxicity analyses were also performed in order to assess whether any observed antiviral effects were due to a general effect on cell viability. This analysis was performed on 96-well plates seeded at the same time and with the identical pool of stock cells used for the antiviral assay described in Example 1. Each compound (celgosivir and ADV) or combination of compounds was tested at four different concentrations in triplicate. Cellular uptake of neutral red dye was used to determine the relative level of toxicity 24 hours after the final treatment. A cell that is viable will take up the neutral red dye, while a dead cell will not. The absorbance of internalized dye at 510 nm (A₅₁₀) was used for quantitative analysis. Values are presented as a percentage of the average A₅₁₀ values (±standard deviation, SD) in 9 separate cultures of untreated cells on the same plate.

TABLE 4
Cytotoxicity - Neutral Red Dye Uptake (% of Control)

| Compound/Dose | 1000 μM | 300 μM | 100 μM | 30 μM |
|---|---|---|---|---|
| Celgosivir | 101% ± 1 | 102% ± 2 | 100% ± 2 | 102% ± 2 |
| ADV | 10% ± 1 | 73% ± 2 | 98% ± 1 | 100% ± 2 |
| Celgosivir (50:1)* | 98% ± 1 | 101% ± 1 | 99% ± 1 | 101% ± 2 |
| Celgosivir (17:1)* | 100% ± 1 | 99% ± 1 | 101% ± 2 | 100% ± 1 |
| Celgosivir (5:1)* | 99% ± 1 | 101% ± 2 | 100% ± 2 | 102% ± 2 |

*Combination treatment of celgosivir + ADV (molar ratio) − the μM dose value in the top row refers to celgosivir (e.g., Celgosivir (50:1) = 1000 μM celgosivir:20 μM ADV). Also indicates the highest tested concentration and that no cytotoxic effects were observed.

The percentage of dye uptake in control (untreated) cell cultures in this experiment was 100%×3 (i.e., values below 100 indicate cytotoxicity). Table 4 shows that neither celgosivir nor any combination of celgosivir with ADV at the concentrations tested affected cell viability (i.e., celgosivir was non-toxic at the concentrations tested). The toxicity pattern of the combination celgosivir+ADV was similar to, and consistent with, the single drug treatment results.

From the cytotoxicity data, a CC₅₀ can be calculated—this is a measure of cytotoxicity and indicates the drug concentration at which a 2-fold reduction in neutral red dye uptake occurs as compared to the average levels in untreated cultures. The EC₉₀ and CC₅₀ values were used to calculate a Selectivity Index (SI, CC₅₀/EC₉₀) because at least a 3-fold reduction in HBV levels is considered as having achieved statistical significance in this assay (see Korba & Gerin, Antiviral Res. 19:55-70,1992).

TABLE 5
Selectivity Index of Celgosivir

| | | | | Selectivity Index |
|---|---|---|---|---|
| Compound | CC₅₀ (μM) | EC₅₀ (μM) | EC₉₀ (μM) | (CC₅₀/EC₉₀) |
| 3TC | 2339 ± 89 | 0.058 ± 0.004 | 0.177 ± 0.015 | 11,709 |
| ADV | 468 ± 17 | 1.0 ± 0.1 | 3.0 ± 0.1 | 162 |
| ADV (50:1) † | 468^{♦} | 0.236 ± 0.027 | 0.867 ± 0.065 | 540^{♦} |
| ADV (17:1) | 468^{♦} | 0.309 ± 0.017 | 1.3 ± 0.2 | 360^{♦} |
| ADV (5:1) | 468^{♦} | 1.0 ± 0.1 | 4.4 ± 0.4 | 106^{♦} |
| Celgosivir | >1000* | 24 ± 1.2 | 147 ± 15 | >6.8 |
| Celgosivir (50:1) †* | >1000* | 12 ± 1.3 | 43 ± 3.2 | >23 |
| Celgosivir (17:1)* | >1000* | 5.1 ± 0.3 | 23 ± 2.0 | >44 |
| Celgosivir (5:1)* | >1000* | 5.6 ± 0.6 | 22 ± 2.2 | >45 |

† Indicates combination treatments of celgosivir with ADV as described for Table 4. The CC₅₀, EC₅₀, and EC₉₀ values are for the drug named.
^{♦}As is shown in Table 4, no toxicity was seen for ADV at the levels tested in this assay (50:1, 20 μM; 17:1, 60 μM; 5:1, 200 μM). Based on this, it is assumed that the ADV CC₅₀ in the combination treatment will be essentially identical to the ADV CC₅₀ in the monotherapy. In view of this assumption, the calculated SIs are only predictions.
*Indicates the highest concentration tested and that no cytotoxic effects were observed.

A drug is predicted to be more efficacious and less toxic as the SI value increases. Table 5 shows that celgosivir has a favorable SI value, although the concentration capable of causing toxicity has yet to be identified. Moreover, it appears that celgosivir may even reduce the toxicity of ADV because the ADV EC₉₀ is reduced in the presence of celgosivir.

### Example 3

### Synergism of Celgosivir Combined with Adefovir Dipivoxil

An exemplary embodiment of the instant disclosure, a glucosidase inhibitor (celgosivir) in combination with an agent that alters Hepadnaviridae replication (adefovir dipivoxil) was used against HBV in an antiviral assay as described in Example 1 to determine the potential of a synergistic interaction. Analysis of synergism, additivity, or antagonism was determined by analysis of the data using the CalcuSyn™ program (Biosoft, Inc.). In one analysis, the Fractional Effect (fraction of virus affected, Fa, i.e., the antiviral effect—that is, if Fa has a value of 0.2 or 0.99, then the viral load was reduced 20% or 99%, respectively) was plotted against the Combination Index (CI). As set forth above, a CI greater than 1.0 indicates antagonism, a CI of 1.0 indicates additivity, and a CI of less than 1.0 indicates synergism. Evaluations of synergy, additivity (summation), or antagonism at different levels of virus inhibition are provided in FIG. 1. The Fa-CI plots are generally very useful in determining drug interactions, especially since a Monte Carlo analysis was included, which provides a measure of statistical significance (the triple lines on the plots represent the median value (middle line) with the upper and lower lines showing the SD, i.e., median values ±1.96 SDs, which are shown relative to the CI=1 line).

In another analysis, isobolograms provide an excellent secondary measure of drug interactions. For these plots, lines are drawn to show the theoretical ED₅₀, ED₇₅, and ED₉₀ values that the combined drugs would demonstrate in an additive interaction (i.e., values for additive interactions based on the results of monotherapy treatment). The actual ED₅₀, ED₇₅, and ED₉₀ values (μM) for the celgosivir+ADV combination treatments are displayed as single points. The single point values that fall to the right of the theoretical additive lines indicate antagonism and values to the left indicate synergy (see FIG. 2).

Overall, celgosivir had a surprising increase in potency against HBV when combined with ADV and, unexpectedly, increased the apparent potency of ADV (see Table 2, FIGS 1A, 1B, 2A, and 2B). A weak degree of antagonism was observed in the combination containing the lowest concentration of celgosivir relative to ADV (see FIGS. 1C and 2C; molar ratio of celgosivir to ADV of 5:1). However, synergistic interactions were observed in the combinations containing higher concentrations of celgosivir relative to ADV (see FIGS. 1A, 1B, 2A, and 2B; molar ratios of celgosivir to ADV of 50:1 and 17:1). In FIG. 1B (17:1 molar ratio), no matter what fraction of HBV is affected by the combination of celgosivir with ADV (Fa=0.1-0.99 or 10-99% reduction in viral load), this particular molar ratio combination shows a strong synergistic interaction. Similarly, the FIG. 2B isobologram shows a synergistic interaction between celgosivir and ADV at an ED₅₀, ED₇₅, and ED₉₀ (i.e., the data point is to the left of the additivity line).

### Example 4

### Anti-HBV Activity of Castanospermine Esters in a Woodchuck Animal Model

The activity of castanospermine ester derivatives alone (such as celgosivir) or in combination with an agent that alters Hepadnaviridae replication, such as a nucleoside analog (e.g., 3TC, ADV, entecavir, clevudine, β-L-nucleosides, 2′,3′-dideoxy-2′,3′-didehydro-β-L-fluorocytidine, tenofovir disoproxil fumarate, etc.; for other exemplary nucleoside analogs see Karayiannis, J. Antimicrob. Chemother. 51:761, 2003) or an agent that alters immune function against Hepadnaviridae (such as IFN-α, pegylated IFN-α, IFN-β, IFN-γ) is examined in a woodchuck model of hepatitis B virus infection (see, e.g., Korba et al., Hepatology 23:958, 1996; Menne & Tennant, Nature Med. 10:1125, 1999; Menne et al., J. Virol. 76:1769, 2002; U.S. Pat. No. 6,878,364).

Briefly, eight groups of four woodchucks each with persistent woodchuck hepatitis virus (WHV) infection (i.e., experimentally infected with WHV during the first week of life) are treated orally with (1) castanospermine alone, (2) a castanospermine ester, such as celgosivir, alone, (3) a nucleoside analog alone, such as ADV or clevudine, (4) an agent that alters immune function against Hepadnaviridae alone, such as pegylated IFN-α or IFN-α; or (4) any combination thereof. The woodchucks are WHsAg positive at the time the study is initiated. Woodchucks in each group can be stratified on the basis of gender, body weight, and age. After treatment with the drugs or drug combinations, woodchucks are administered 4 to 5 mL semisynthetic liquid woodchuck diet to insure complete ingestion of the drugs. The antiviral activity of the individual drugs and combinations can be assessed by measuring serum WHV DNA during treatment, and comparing the results of treated groups to placebo treated controls.

Woodchucks are anesthetized (50 mg/kg ketamine, 5 mg/kg zylazine), weighed, and blood samples obtained prior to initial treatment, at weekly intervals during the six week period of treatment, and at 1, 2 and 4 weeks following treatment. Woodchuck serum is harvested and divided into several aliquots. One aliquot is used for detection of WHV DNA by dot blot hybridization and for detection of the WHsAg by ELISA. Complete blood counts (CBCs) and clinical biochemical profiles are obtained before drug treatment and after drug treatment is complete. A second aliquot is stored as an archive sample, while other aliquots of serum are used for drug analysis and specific WHV DNA analyses, as described herein.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of treating or preventing a Hepadnaviridae infection, comprising administering to a subject a glucosidase inhibitor or pharmaceutically acceptable salts thereof, wherein the glucosidase inhibitor has the following structural formula (I): 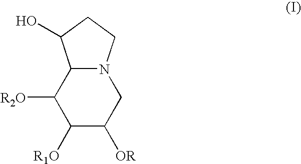
wherein R, R₁ and R₂ are independently hydrogen, C₁₋₁₄ alkanoyl, C₂₋₁₄ alkenoyl, cyclohexanecarbonyl, C₁₋₈ alkoxyacetyl, 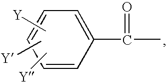
naphthalenecarbonyl optionally substituted with a methyl or halogen; phenyl(C₂₋₆ alkanoyl), wherein the phenyl is optionally substituted with a methyl or halogen; cinnamoyl; pyridinecarbonyl optionally substituted with a methyl or halogen; dihydropyridine carbonyl optionally substituted with a C₁₋₁₀ alkyl; thiophenecarbonyl optionally substituted with methyl or halogen; or furancarbonyl optionally substituted by a methyl or halogen;
Y is hydrogen, C₁₋₄ alkyl, C₁₋₄ alkoxy, halogen, trifluoromethyl, C₁₋₄ alkylsulfonyl, C₁₋₄ alkylmercapto, cyano or dimethylamino;
Y′ is hydrogen, C₁₋₄ alkyl, C₁₋₄ alkoxy, halogen or it is combined with Y to give 3,4-methylenedioxy;
Y″ is hydrogen, C₁₋₄ alkyl, C₁₋₄ alkoxy or halogen; and
wherein at least one, but not more than two, of R, R₁ and R₂ is hydrogen:

2. The method according to claim 1 wherein the glucosidase inhibitor structural formula (I) has the following stereochemistry:

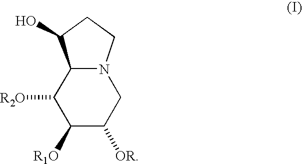

3. The method according to claim 1 wherein R, R₁ and R₂ are each independently hydrogen, C₁₋₁₀ alkanoyl, C₂₋₁₀ alkenoyl, C₁₋₈ alkoxyacetyl; or 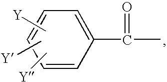
wherein Y is hydrogen, C₁₋₄ alkyl, C₁₋₄ alkoxy, halogen, trifluoromethyl, C₁₋₄ alkylsulfonyl, C₁₋₄ alkylmercapto, cyano or dimethylamino;
Y′ is hydrogen, C₁₋₄ alkyl, C₁₋₄ alkoxy, halogen or it is combined with Y to give 3,4-methylenedioxy;
Y″ is hydrogen, C₁₋₄ alkoxy or halogen; and
wherein at least one, but not more than two, of R, R₁ and R₂ is hydrogen.

4. The method according to claim 1 wherein R, R₁ and R₂ are each independently hydrogen, C₁₋₈ alkanoyl, C₂₋₈ alkenoyl, C₁₋₈ alkoxy-acetyl, or a benzoyl optionally substituted with an alkyl or halogen; and
wherein at least one, but not more than two, of R, R₁ and R₂ is hydrogen.

5. The method according to claim 1 wherein R, R₁ and R₂ are each independently hydrogen, C₁₋₈ alkanoyl, C₂₋₈ alkenoyl, C₁₋₈ alkoxy-acetyl, or a benzoyl optionally substituted with a methyl, bromo, chloro, or fluoro group; and
wherein at least one, but not more than two, of R, R₁ and R₂ is hydrogen.

6. The method according to claim 1 wherein R₁ is a C₁₋₈ alkanoyl, C₂₋₁₀ alkenoyl, C₁₋₈ alkoxy-acetyl, or a benzoyl optionally substituted with an alkyl or halogen group.

7. The method according to claim 1 wherein R₁ is a C₁₋₈ alkanoyl, C₂₋₈ alkenoyl, C₁₋₈ alkoxyacetyl, or a benzoyl optionally substituted with a methyl, bromo, chloro, or fluoro group.

8. The method according to claim 1 wherein the glucosidase inhibitor is:
(a) [1S-(1α,6β,7α,8β,8aβ)]-octahydro-1,6,7,8-indolizinetetrol 6-benzoate;
(b) [1S-(1α,6β,7α,8β,8aβ)]-octahydro-1,6,7,8-indolizinetetrol 7-benzoate;
(c) [1S-(1α,6β,7α,8β,8aβ)]-octahydro-1,6,7,8-indolizinetetrol 6-(4-methylbenzoate);
(d) [1S-(1α,6β,7α,8β,8aβ)]-octahydro-1,6,7,8-indolizinetetrol 7-(4-bromobenzoate);
(e) [1S-(1α,6β,7α,8β,8aβ)]-octahydro-1,6,7,8-indolizinetetrol 6,8-dibutanoate;
(f) [1S-(1α,6β,7α,8β,8aβ)]-octahydro-1,6,7,8-indolizinetetrol 6-butanoate;
(g) [1S-(1α,6β,7α,8β,8aβ)]-octahydro-1,6,7,8-indolizinetetrol 6-(2-furancarboxylate);
(h) [1S-(1α,6β,7α,8β,8aβ)]-octahydro-1,6,7,8-indolizinetetrol 7-(2,4-dichlorobenzoate);
(i) [1S-(1α,6β,7α,8β,8aβ)]-octahydro-1,6,7,8-indolizinetetrol 6-(3-hexenoate);
(j) [1S-(1α,6β,7α,8β,8aβ)]-octahydro-1,6,7,8-indolizinetetrol 6-octanoate;
(k) [1S-(1α,6β,7α,8β,8aβ)]-octahydro-1,6,7,8-indolizinetetrol 6-pentanoate;
(l) an O-pivaloyl ester;
(m) a 2-ethyl-butyryl ester;
(n) a 3,3-dimethylbutyryl ester;
(o) a cyclopropanoyl ester;
(p) a 4-methoxybenzoate ester;
(q) a 2-aminobenzoate ester; or
(r) a mixture of at least two of (a)-(q).

9. The method according to claim 1 wherein the glucosidase inhibitor is [1S-(1α,6β,7α,8β,8aβ)]-octahydro-1,6,7,8-indolizinetetrol 6-benzoate.

10. The method according to claim 1 wherein the glucosidase inhibitor is [1S-(1α,6β,7α,8β,8aβ)]-octahydro-1,6,7,8-indolizinetetrol 6-butanoate.

11. The method according to claim 1 wherein the glucosidase inhibitor is [1S-(1α,6β,7α,8β,8aβ)]-octahydro-1,6,7,8-indolizinetetrol 6-pentanoate.

12. The method according to claim 1 wherein the glucosidase inhibitor is [1S-(1α,6β,7α,8β,8aβ)]-octahydro-1,6,7,8-indolizinetetrol 6-(2-furancarboxylate).

13. The method according to claim 1 wherein the subject is a human.

14. The method according to claim 1 wherein the Hepadnaviridae is a member of the genus Avihepadnavirus.

15. The method according to claim 1 wherein the Hepadnaviridae is a member of the genus Orthohepadnavirus.

16. The method of claim 15 wherein the Hepadnaviridae is hepatitis B virus (HBV).

17. A method for treating or preventing a Hepadnaviridae infection, comprising administering to a subject a combination of an agent that alters immune function, and a glucosidase inhibitor and pharmaceutically acceptable salts thereof, wherein the glucosidase inhibitor has a structure according to formula (I): 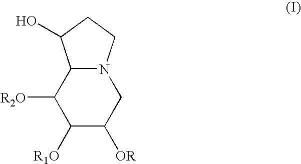
wherein R, R₁ and R₂ are independently selected from hydrogen, C₁₋₁₄ alkanoyl, C₂₋₁₄ alkenoyl, cyclohexanecarbonyl, C₁₋₈ alkoxyacetyl, 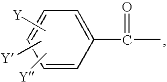
naphthalenecarbonyl optionally substituted with a methyl or halogen; phenyl(C₂₋₆ alkanoyl), wherein the phenyl is optionally substituted with a methyl or halogen; cinnamoyl; pyridinecarbonyl optionally substituted with a methyl or halogen; dihydropyridine carbonyl optionally substituted with a C₁₋₁₀ alkyl; thiophenecarbonyl optionally substituted with methyl or halogen; or furancarbonyl optionally substituted by a methyl or halogen;
Y is hydrogen, C₁₋₄ alkyl, C₁₋₄ alkoxy, halogen, trifluoromethyl, C₁₋₄ alkylsulfonyl, C₁₋₄ alkylmercapto, cyano or dimethylamino;
Y′ is hydrogen, C₁₋₄ alkyl, C₁₋₄ alkoxy, halogen or it is combined with Y to give 3,4-methylenedioxy;
Y″ is hydrogen, C₁₋₄ alkyl, C₁₋₄ alkoxy or halogen; and
wherein at least one, but not more than two, of R, R₁ and R₂ is hydrogen.

18. The method according to claim 17 wherein the glucosidase inhibitor structural formula (I) has the following stereochemistry:

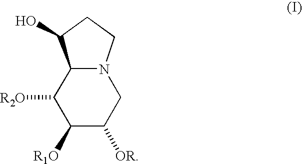

19. The method according to claim 17 wherein R, R₁ and R₂ are each independently hydrogen, C₁₋₁₀ alkanoyl, C₂₋₁₀ alkenoyl, C₁₋₈ alkoxyacetyl; or 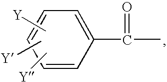
wherein Y is hydrogen, C₁₋₄ alkyl, C₁₋₄ alkoxy, halogen, trifluoromethyl, C₁₋₄ alkylsulfonyl, C₁₋₄ alkylmercapto, cyano or dimethylamino;
Y′ is hydrogen, C₁₋₄ alkyl, C₁₋₄ alkoxy, halogen or it is combined with Y to give 3,4-methylenedioxy;
Y″ is hydrogen, C₁₋₄ alkoxy or halogen; and
wherein at least one, but not more than two, of R, R₁ and R₂ is hydrogen.

20. The method according to claim 17 wherein R, R₁ and R₂ are each independently hydrogen, C₁₋₈ alkanoyl, C₂₋₈ alkenoyl, C₁₋₈ alkoxy-acetyl, or a benzoyl optionally substituted with an alkyl or halogen; and
wherein at least one, but not more than two, of R, R₁ and R₂ is hydrogen.

21. The method according to claim 17 wherein R, R₁ and R₂ are each independently hydrogen, C₁₋₈ alkanoyl, C₁₋₈ alkenoyl, C₁₋₈ alkoxy-acetyl, or a benzoyl optionally substituted with a methyl, bromo, chloro, or fluoro group; and
wherein at least one, but not more than two, of R, R₁ and R₂ is hydrogen.

22. The method according to claim 17 wherein R₁ is a C₁₋₈ alkanoyl, C₂₋₁₀ alkenoyl, C₁₋₈ alkoxy-acetyl, or a benzoyl optionally substituted with an alkyl or halogen group.

23. The method according to claim 17 wherein R₁ is a C₁₋₈ alkanoyl, C₂₋₈ alkenoyl, C₁₋₈ alkoxyacetyl, or a benzoyl optionally substituted with a methyl, bromo, chloro, or fluoro group.

24. The method according to claim 17 wherein the glucosidase inhibitor is:
(a) [1S-(1α,6β,7α,8β,8aβ)]-octahydro-1,6,7,8-indolizinetetrol 6-benzoate;
(b) [1S-(1α,6β,7α,8β,8aβ)]-octahydro-1,6,7,8-indolizinetetrol 7-benzoate;
(c) [1S-(1α,6β,7α,8β,8aβ)]-octahydro-1,6,7,8-indolizinetetrol 6-(4-methylbenzoate);
(d) [1S-(1α,6β,7α,8β,8aβ)]-octahydro-1,6,7,8-indolizinetetrol 7-(4-bromobenzoate);
(e) [1S-(1α,6β,7α,8β,8aβ)]-octahydro-1,6,7,8-indolizinetetrol 6,8-dibutanoate;
(f) [1S-(1α,6β,7α,8β,8aβ)]-octahydro-1,6,7,8-indolizinetetrol 6-butanoate;
(g) [1S-(1α,6β,7α,8β,8aβ)]-octahydro-1,6,7,8-indolizinetetrol 6-(2-furancarboxylate);
(h) [1S-(1α,6β,7α,8β,8aβ)]-octahydro-1,6,7,8-indolizinetetrol 7-(2,4-dichlorobenzoate);
(i) [1S-(1α,6β,7α,8β,8aβ)]-octahydro-1,6,7,8-indolizinetetrol 6-(3-hexenoate);
(j) [1S-(1α,6β,7α,8β,8aβ)]-octahydro-1,6,7,8-indolizinetetrol 6-octanoate;
(k) [1S-(1α,6β,7α,8β,8aβ)]-octahydro-1,6,7,8-indolizinetetrol 6-pentanoate;
(1) an O-pivaloyl ester;
(m) a 2-ethyl-butyryl ester;
(n) a 3,3-dimethylbutyryl ester;
(o) a cyclopropanoyl ester;
(p) a 4-methoxybenzoate ester;
(q) a 2-aminobenzoate ester; or
(r) a mixture of at least two of (a)-(q).

25. The method according to claim 17 wherein the glucosidase inhibitor is [1S-(1α,6β,7α,8β,8aβ)]-octahydro-1,6,7,8-indolizinetetrol 6-benzoate.

26. The method according to claim 17 wherein the glucosidase inhibitor is [1S-(1α,6β,7α,8β,8aβ)]-octahydro-1,6,7,8-indolizinetetrol 6-butanoate.

27. The method according to claim 17 wherein the glucosidase inhibitor is [1S-(1α,6β,7α,8β,8aβ)]-octahydro-1,6,7,8-indolizinetetrol 6-pentanoate.

28. The method according to claim 17 wherein the glucosidase inhibitor is [1S-(1α,6β,7α,8β,8aβ)]-octahydro-1,6,7,8-indolizinetetrol 6-(2-furancarboxylate).

29. The method according to claim 17 wherein the subject is a human.

30. The method according to claim 17 wherein the agent that alters immune function is an interferon.

31. The method according to claim 30 wherein the interferon is interferon-α.

32. The method according to claim 30 wherein the interferon-α is pegylated.

33. The method according to claim 17 wherein the agent that alters immune function is administered before the glucosidase inhibitor.

34. The method according to claim 17 wherein the glucosidase inhibitor is administered before the agent that alters immune function.

35. The method according to claim 17 wherein the glucosidase inhibitor and the agent that alters immune function are admixed as a single composition and administered simultaneously.

36. The method according to claim 17 wherein the Hepadnaviridae is a member of the genus Avihepadnavirus.

37. The method according to claim 17 wherein the Hepadnaviridae is a member of the genus Orthohepadnavirus.

38. The method according to claim 37 wherein the Hepadnaviridae is HBV.

39. The method according to claim 17 wherein the glucosidase inhibitor and the agent that alters immune function further comprise a pharmaceutically acceptable carrier, diluent or excipient.

40. A method for treating a Hepadnaviridae infection, comprising administering to a subject an agent that alters viral replication, and a glucosidase inhibitor and pharmaceutically acceptable salts thereof, wherein the glucosidase inhibitor has a structure according to formula (I): 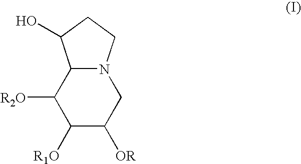
wherein R, R₁ and R₂ are independently selected from hydrogen, C₁₋₁₄ alkanoyl, C₂₋₁₄ alkenoyl, cyclohexanecarbonyl, C₁₋₈ alkoxyacetyl, 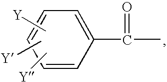
naphthalenecarbonyl optionally substituted with a methyl or halogen; phenyl(C₂₋₆ alkanoyl), wherein the phenyl is optionally substituted with a methyl or halogen; cinnamoyl; pyridinecarbonyl optionally substituted with a methyl or halogen; dihydropyridine carbonyl optionally substituted with a C₁₋₁₀ alkyl; thiophenecarbonyl optionally substituted with methyl or halogen; or furancarbonyl optionally substituted by a methyl or halogen;
Y is hydrogen, C₁₋₄ alkyl, C₁₋₄ alkoxy, halogen, trifluoromethyl, C₁₋₄ alkylsulfonyl, C₁₋₄ alkylmercapto, cyano or dimethylamino;
Y′ is hydrogen, C₁₋₄ alkyl, C₁₋₄ alkoxy, halogen or it is combined with Y to give 3,4-methylenedioxy;
Y″ is hydrogen, C₁₋₄ alkyl, C₁₋₄ alkoxy or halogen;
wherein at least one, but not more than two, of R, R₁ and R₂ is hydrogen.

41. The method according to claim 40 wherein the glucosidase inhibitor structural formula (I) has the following stereochemistry:

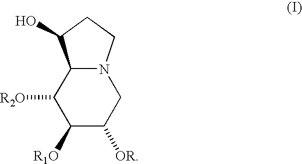

42. The method according to claim 40 wherein R, R₁ and R₂ are each independently hydrogen, C₁₋₁₀ alkanoyl, C₂₋₁₀ alkenoyl, C₁₋₈ alkoxyacetyl; or 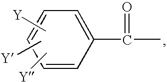
wherein Y is hydrogen, C₁₋₄ alkyl, C₁₋₄ alkoxy, halogen, trifluoromethyl, C₁₋₄ alkylsulfonyl, C₁₋₄ alkylmercapto, cyano or dimethylamino;
Y′ is hydrogen, C₁₋₄ alkyl, C₁₋₄ alkoxy, halogen or it is combined with Y to give 3,4-methylenedioxy;
Y″ is hydrogen, C₁₋₄ alkoxy or halogen; and
wherein at least one, but not more than two, of R, R₁ and R₂ is hydrogen.

43. The method according to claim 40 wherein R, R₁ and R₂ are each independently hydrogen, C₁₋₈ alkanoyl, C₂₋₈ alkenoyl, C₁₋₈ alkoxy-acetyl, or a benzoyl optionally substituted with an alkyl or halogen; and
wherein at least one, but not more than two, of R, R₁ and R₂ is hydrogen.

44. The method according to claim 40 wherein R, R₁ and R₂ are each independently hydrogen, C₁₋₈ alkanoyl, C₂₋₈ alkenoyl, C₁₋₈ alkoxy-acetyl, or a benzoyl optionally substituted with a methyl, bromo, chloro, or fluoro group; and
wherein at least one, but not more than two, of R, R₁ and R₂ is hydrogen.

45. The method according to claim 40 wherein R₁ is a C₁₋₈ alkanoyl, C₂₋₁₀ alkenoyl, C₁₋₈ alkoxy-acetyl, or a benzoyl optionally substituted with an alkyl or halogen group.

46. The method according to claim 40 wherein R₁ is a C₁₋₈ alkanoyl, C₂₋₈ alkenoyl, C₁₋₈ alkoxyacetyl, or a benzoyl optionally substituted with a methyl, bromo, chloro, or fluoro group.

47. The method according to claim 40 wherein the glucosidase inhibitor is:
(a) [1S-(1α,6β,7α,8β,8aβ)]-octahydro-1,6,7,8-indolizinetetrol 6-benzoate;
(b) [1S-(1α,6β,7α,8β,8aβ)]-octahydro-1,6,7,8-indolizinetetrol 7-benzoate;
(c) [1S-(1α,6β,7α,8β,8aβ)]-octahydro-1,6,7,8-indolizinetetrol 6-(4-methylbenzoate);
(d) [1S-(1α,6β,7α,8β,8aβ)]-octahydro-1,6,7,8-indolizinetetrol 7-(4-bromobenzoate);
(e) [1S-(1α,6β,7α,8β,8aβ)]-octahydro-1,6,7,8-indolizinetetrol 6,8-dibutanoate;
(f) [1S-(1α,6β,7α,8β,8aβ)]-octahydro-1,6,7,8-indolizinetetrol 6-butanoate;
(g) [1S-(1α,6β,7α,8β,8aβ)]-octahydro-1,6,7,8-indolizinetetrol 6-(2-furancarboxylate);
(h) [1S-(1α,6β,7α,8β,8aβ)]-octahydro-1,6,7,8-indolizinetetrol 7-(2,4-dichlorobenzoate);
(i) [1S-(1α,6β,7α,8β,8aβ)]-octahydro-1,6,7,8-indolizinetetrol 6-(3-hexenoate);
(j) [1S-(1α,6β,7α,8β,8aβ)]-octahydro-1,6,7,8-indolizinetetrol 6-octanoate;
(k) [1S-(1α,6β,7α,8β,8aβ)]-octahydro-1,6,7,8-indolizinetetrol 6-pentanoate;
(1) an O-pivaloyl ester;
(m) a 2-ethyl-butyryl ester;
(n) a 3,3-dimethylbutyryl ester;
(o) a cyclopropanoyl ester;
(p) a 4-methoxybenzoate ester;
(q) a 2-aminobenzoate ester; or
(r) a mixture of at least two of (a)-(q).

48. The method according to claim 40 wherein the glucosidase inhibitor is [1S-(1α,6β,7α,8β,8aβ)]-octahydro-1,6,7,8-indolizinetetrol 6-benzoate.

49. The method according to claim 40 wherein the glucosidase inhibitor is [1S-(1α,6β,7α,8β,8aβ)]-octahydro-1,6,7,8-indolizinetetrol 6-butanoate.

50. The method according to claim 40 wherein the glucosidase inhibitor is [1S-(1α,6β,7α,8β,8aβ)]-octahydro-1,6,7,8-indolizinetetrol 6-pentanoate.

51. The method according to claim 40 wherein the glucosidase inhibitor is [1S-(1α,6β,7α,8β,8aβ)]-octahydro-1,6,7,8-indolizinetetrol 6-(2-furancarboxylate).

52. The method according to claim 40 wherein the subject is a human.

53. The method according to claim 40 wherein the agent that alters viral replication is adefovir dipivoxil.

54. The method according to claim 40 wherein the agent that alters viral replication is lamivudine.

55. The method according to claim 40 wherein the agent that alters viral replication is clevudine.

56. The method according to claim 40 wherein the agent that alters viral replication is at least one of adefovir dipivoxil, lamivudine, clevudine and ribavirin.

57. The method according to claim 40 wherein the agent that alters viral replication is administered before the glucosidase inhibitor.

58. The method according to claim 40 wherein the glucosidase inhibitor is administered before the agent that alters viral replication.

59. The method according to claim 40 wherein the glucosidase inhibitor and the agent that alters viral replication are admixed as a single composition and administered simultaneously.

60. The method according to claim 40 wherein the Hepadnaviridae is a member of the genus Avihepadnavirus.

61. The method according to claim 40 wherein the Hepadnaviridae is a member of the genus Orthohepadnavirus.

62. The method according to claim 40 wherein the Hepadnaviridae is HBV.

63. The method according to claim 40 wherein the glucosidase inhibitor and the agent that alters immune function further comprise a pharmaceutically acceptable carrier, diluent or excipient.

64. A method for treating or preventing Hepadnaviridae infection comprising a glucosidase inhibitor as defined in claim 1 in combination with an agent selected from:
(a) a compound that inhibits infection of cells by Hepadnaviridae;
(b) a compound that inhibits the release of viral RNA from the viral capsid or the function of Hepadnaviridae gene products;
(c) a compound that alters Hepadnaviridae replication;
(d) a compound that alters immune function against Hepadnaviridae; and
(e) a compound that alters symptoms of a Hepadnaviridae infection.

65. The method according to claim 64 wherein the compound that alters immune function is an interferon.

66. The method according to claim 65 wherein the interferon is interferon-α or pegylated interferon-α.

67. The method according to claim 64 wherein the compound that alters Hepadnaviridae viral replication is adefovir dipivoxil, clevudine, lamivudine or ribavirin.

68. The method according to claim 64 wherein the Hepadnaviridae is a member of the genus Orthohepadnavirus.

69. The method according to claim 64 wherein the Hepadnaviridae is HBV.

70. The method according to claim 64 wherein the composition further comprises a pharmaceutically acceptable diluent, carrier or excipient.
